(19) United States
(12) Patent Application Publication
(10) Pub. No.: US 2012/0083683 A1
(43) Pub. Date: Apr. 5, 2012

(54) DIAGNOSIS APPARATUS

(75) Inventor: Yoshihiko Kuwabara, Hamamatsu-shi (JP)

(73) Assignee: National University Corp. Shizuoka University, Shizuoka-shi, Shizuoka (JP)

(21) Appl. No.: 13/377,264

(22) PCT Filed: Jun. 10, 2010
(86) PCT No.: PCT/JP2010/059855

§ 371 Date: Dec. 9, 2011

(30) Foreign Application Priority Data
Jun. 10, 2009 (JP) 2009-139664

(51) Int. Cl. A61B 5/05

(52) U.S. Cl. 600/407

(57) ABSTRACT

A diagnosis apparatus encompasses a probe array (1, 2) made of material having electromagnetic characteristic identical to the electromagnetic characteristic of a target object, the probe array (1, 2) embraces a receptacle 1 having a semispherical inner wall surface, and a plurality of antennas 2, arranged along the inner wall surface, the plurality of antennas 2 carry out electrical measurement of a target region in the target object, a fixing mechanism configured to cover whole of the target region with the probe array (1, 2), bringing skin of the target region into close contact with the inner wall surface, so as to fix a relative position between the target region and the probe array (1, 2), and a measuring, controlling and analyzing mechanism configured to control the plurality of antennas 2, to execute the electrical measurement, and to analyze a data based on the electrical measurement so as to detect an abnormal cell in the target region. The diagnosis apparatus has a high contrast and a high resolution and does not suffer from any X-ray exposure and is low in diagnosis cost and safe, sure, comfortable and high in speed and high in reliability.

### TECHNICAL FIELD

The present invention pertains to a diagnosis apparatus used to diagnose abnormal cells such as early-stage breast-cancers and the like.

### Background Art

In recent years, a research for detecting early-stage breast-cancers via a microwave imaging has been eagerly performed. Principle for detecting cancers is based upon remarkable differences of electromagnetic parameters (dielectric constant and conductivity) between peripheral (fatty) tissues and a cancer tissue. That is, the principle for detecting cancers is based upon a fact that the backscattered wave from the cancer tissue is larger than the backscattered wave from normal tissues. As examples of the imaging scheme based on the microwave imaging, there are an ultra-wideband (UWB) radar approach (see non-patent literatures (NPLs) 1 and 2) and a tomography approach (see NPL 3).

In the scheme disclosed in NPL 1, by using a monostatic radar, wide band pulses are emitted to a breast from many directions and received in the same direction, and a three-dimensional backscattered electric field distribution is determined by a space-time directivity synthesis. In the scheme disclosed in NPL 2, by using a multi-static radar, the responses to the pulse emitted from a certain direction is received by a plurality of antennas located at different positions. Each time the emission direction is changed, it is received by the plurality of antennas. A Capon architecture, known as an adaptive beamforming algorism, is used to improve the resolution of the schemes disclosed in NPL 1 by carrying out the directivity synthesis so that the responses except a corresponding pixel become zero. In the scheme disclosed in NPL 3, the electromagnetic waves in a narrow band are emitted to the breast and received by the plurality of antennas located at different positions. The reception response to a certain transmission signal can be calculated on the basis of Maxwell equation, when a propagation model (for example, a model implemented by the skin, fatty tissues, a lacteal gland and a cancer) is given. In the scheme disclosed in NPL 3, the propagation model is presumed from the received signal, by solving an inverse problem

In patent literature (PTL) 1 pertaining to the tomography that uses the electromagnetic waves, in order to avoid the antenna from becoming a larger size, ascribable to a size of the wavelength of the electromagnetic wave, an electromagnetic coil is used instead of the antenna employed in the scheme disclosed in NPL 3. Then, the other features of the apparatus are equal to the configurations disclosed in NPL 3.

The reflection of microwaves from the skin is large, and the permeation quantity of microwaves into the tissue is small. For those reasons, in all of the schemes disclosed in NPLs 1, 2 and 3, as illustrated in FIG. 24, an antenna 51 and a breast 53 are immersed into a matching medium 52 having characteristics close to the electromagnetic parameter of the normal issue in the breast so that impedance matching is carried out, thereby increasing the permeation quantity of the electromagnetic wave into the issue. Here, a person under diagnostic-test shall lie in the prone posture (or face-down posture), and diagnosis is performed on the posture in which her breasts droop. The reflection of the electromagnetic wave from the skin cannot be perfectly removed even by using the matching medium. In particular, in the schemes disclosed in NPLs 1 and 2 using the UWB radar, since the response from the cancer is very small, the response signal is hidden behind the signals by the reflection of the electromagnetic waves from the skin. Even the methodology disclosed in NPL 3 requires preliminary knowledge with regard to the three-dimensional shape of the target object. In order to remove artifacts such as the reflection from the skin and the like, it is effective to average the plurality of response signals in which the distance relations between the transmitting receiving antennas and the skin are equal and defining the averaged response signal as a calibration signal and then subtracting the calibration signal from the reception signal.

As for the shape of the breast, since the personal difference is large, it is difficult to keep the distance between the antenna and the skin constant. For this reason, it is necessary to measure a distance between the skin of the breast and the antenna and modify the reception signal on the basis of the measured distance.

As for the measurement of the distance between the breast and the antenna, a method of using the UWB radar and a method of using a laser radar are considered. In the scheme disclosed in NPL 4, while the wide band pulses between 1 and 11 GHz are emitted, the antennas are scanned mechanically and spirally, the data measured at 40 locations are obtained and then interpolated at 1000 points, and the three-dimensional shape of the breast is consequently presumed. In the scheme disclosed in NPL 5, the UWB radar and the laser radar are rotationally scanned while their heights are changed, and the three-dimensional shape of the breast is presumed. Then, the fact that the laser radar is high in presumption precision is reported.

In an X-ray mammography serving as a current screening means for the breast cancer, the breast is sandwiched between glass plates and made flat and then imaged. Thus, the pain of a person under diagnostic-test is severe. For this reason, PTL 2 describes a method of placing an X-ray film on one side of a receptacle suited to the breast and emitting the X-ray from the opposite side. Also, PTL 3 describes a method that adjusts the shape of the portion to be imaged, by inserting the breast into the receptacle in order to fix the position of the breast at the time of the X-ray photography and further sucking with a vacuum pump.

Also, PTL 4 describes a method of sticking various sensors (a light, an X-ray, an electromagnetic wave, an ultrasonic wave, a magnetism and an impedance) on the inner side of a rigidity surface and bringing into close contact with the portion to be imaged and then imaging.

### Citation List

Patent Literature

[PTL 1] JP-H06-503731A

[PTL 2] JP-2007-159965A

[PTL 3] JP-2008-220638A

[PTL 4] JP-2009-508539A

[Non-Patent Literature]

[NPL 1] “An Overview of Ultra-Wideband Microwave Imaging via Space-Time Beamforming for Early-Stage Breast Cancer Detection”, IEEE Antennas and Propagation Magazine, Vol. 47, No. 1, 2005, pp. 19-34

[NPL 2] “Microwave Imaging via Adaptive Beam Forming Methods for Breast Cancer Detection”, Journal of Electromagnetic Waves and Applications, Vol. 20, No. 1, 2006, pp. 53-63

[NPL 3] “Nonlinear Microwave Imaging for Breast-Cancer Screening Using Gauss-Newton's Method and the CGLS Inversion Algorithm”, IEEE Transactions on Antennas and Propagation, Vol. 55, No. 8, 2007, pp. 2320-31

[NPL 4] “Estimating the Breast Surface Using UWB Microwave Monostatic Backscatter Measurements”, IEEE Transactions on Biomedical Engineering, Vo. 55, No. 1, 2008, pp. 247-256

[NPL 5] “Comparison of microwave and laser surface detection for microwave imaging systems”, 13th International Symposium on Antenna Technology and Applied Electromagnetics and the Canadian Radio Sciences Meeting, 2009

SUMMARY OF INVENTION

### Technical Problem

The conventional schemes for screening for the early-stage breast-cancer have the following problems. As for schemes recited in PTLs 2 and 3, since an X-ray source is separated from the receptacle for fixing the breast, a positioning mechanism of the receptacle is large in size. The X-ray mammography has defects such as the risk from X-ray exposure and low contrast, and the apparatus scale is large and an X-ray emission engineer is required. Thus, the X-ray mammography is high in cost as the diagnosing means.

As for scheme recited in PTL 4, the application to the ultrasonic wave and impedance CT are assumed, and only the close contact between the sensor and the imaging unit is explained. When an antenna element and the skin are brought into close contact, the impedance characteristics of the antenna is changed, and the reflection loss is increased, and the electromagnetic wave is not transmitted into the imaging unit. Thus, the response signal required to image cannot be obtained.

As for the schemes disclosed in NPLs 1 and 3, with the lack of the resolution, the detection of the early-stage cancer having a size of several millimeters is difficult. In NPL 1, the clinical imaging is not reported, and in NPL 3, only the imaged result of the advanced cancel whose diameter is 4 cm is reported.

The scheme recited in NPL 2 is higher in resolution than the scheme recited in NPL 1. However, the methodology disclosed in NPL 2 includes a parameter that is not uniquely determined in the course of a calculating process. Thus, the improper setting of the parameter results in the failure of the imaging. Also, since the calculation quantity is enormous, it takes a long time to get the final diagnosis image.

In the scheme recited in NPL 3, from a plurality of received signals, the dielectric constant or conductivity distribution in a target region is determined on the basis of the inverse problem. The inverse problem is typically an ill-posed problem, and made proper by using a Tikhonov method, in many cases. The Tikhonov method includes the parameter that cannot be uniquely determined, and the improper setting of the parameter results in the failure of the imaging. Also, an electromagnetic wave propagation analysis is carried out, and in comparison with the solution gotten on the basis of the inverse problem, the best answer is determined. However, since the calculation quantity of the electromagnetic wave propagation analysis is large, it takes a long time to get the final diagnosis image.

Also, in all of the microwave imaging schemes, the matching medium is required. Even if a person under diagnostic-test whose breast is small lies in the prone posture (or face-down posture), the drooping amount of the breast is small, which makes the immersion into the matching medium difficult. As the matching medium is mixed with fats and oils (glycerin and the like), the discomfort feeling when the breast is immersed into the matching medium and the peripheral contamination caused by the droplets of the matching medium are predicted. Also, it is necessary to measure a distance between the breast and the antenna and modify the received signal on the basis of the measured distance. The position of a distance measurement sensor is required to be scanned mechanically and continuously. When the breast is moved, the reliability of the imaged result is dropped, which requires that the breast is fixed during the diagnosis. The scale of the diagnosis apparatus, the discomfort feeling of persons under diagnostic-test and the increase in the diagnosis time are predicted.

In view of the above problems, an object of the present invention is to provide a diagnosis apparatus for detecting abnormal cells via microwave imaging, which has a high contrast and a high resolution and does not suffer from any X-ray exposure and is low in diagnosis cost and safe, sure, comfortable and high in speed and high in reliability.

### Solution to Problem

In order to achieve the above-mentioned object, a first aspect of the present invention inheres in a diagnosis apparatus encompassing (a) a probe array made of material having electromagnetic characteristic identical to the electromagnetic characteristic of a target object, the probe array embraces a receptacle having a semispherical inner wall surface, and a plurality of probes, arranged along the inner wall surface, the plurality of probes carry out electrical measurement of a target region in the target object; (b) a fixing mechanism configured to cover whole of the target region with the probe array, bringing skin of the target region into close contact with the inner wall surface, so as to fix a relative position between the target region and the probe array; and (c) a measuring, controlling and analyzing mechanism configured to control the plurality of probes, to execute the electrical measurement, and to analyze a data based on the electrical measurement so as to detect an abnormal cell in the target region.

### Advantageous Effects of Invention

According to the present invention, it is possible to provide a diagnosis apparatus for detecting abnormal cells via the microwave imaging, which has a high contrast and a high resolution and does not suffer from any X-ray exposure and is low in diagnosis cost and safe, sure, comfortable and high in speed and high in reliability.

### BRIEF DESCRIPTION OF DRAWINGS

FIG. 1 (a) is a diagrammatic view illustrating the outline of a sensor unit of a diagnosis apparatus according to a first embodiment of the present invention;

FIG. 1 (b) is a cross-sectional view describing a structure of a probe that is used in the diagnosis apparatus pertaining to the first embodiment of the present invention;

FIG. 2 is a block diagram describing the structure of the probe pertaining to the first embodiment;

FIG. 3 is a cross-sectional view describing a structure of a probe (antenna) that is used in the diagnosis apparatus pertaining to the first embodiment;

FIG. 4 is a frequency dependence of voltage standing wave ratios in which the probe, used in the diagnosis apparatus pertaining to the first embodiment, is simulated;

FIG. 5 is a block diagram describing an artifact removal method that is used in a diagnosis method pertaining to the first embodiment;

FIG. 6 is an example illustrating a signal obtained by the artifact removal method illustrated in FIG. 5 and a reception signal of one probe.

FIG. 7 is a conceptual view describing the principle of the imaging algorism that is used in the conventional ultrasonic diagnosis apparatus, for a comparison;

FIG. 8 is a block diagram describing an algorism for calculating a backscattered power that is used in the re-construction of a diagnosis image of the diagnosis apparatus pertaining to the first embodiment;

FIG. 9 (a) is an X-Y plan view of a simulation model to indicate the validity of the re-construction algorism of the diagnosis image of the diagnosis apparatus pertaining to the first embodiment;

FIG. 9 (b) is an X-Z plan view of the model illustrated in FIG. 9 (a);

FIG. 10 is a simulation condition to indicate the validity of the re-construction algorism of the diagnosis image of the diagnosis apparatus pertaining to the first embodiment;

FIG. 11 is a simulation model to indicate the validity of the re-construction algorism of the diagnosis image of the diagnosis apparatus pertaining to the first embodiment;

FIG. 12 is a simulation result to indicate the validity of the re-construction algorism of the diagnosis image of the diagnosis apparatus pertaining to the first embodiment;

FIG. 13 is the simulation result to indicate the validity of the re-construction algorism of the diagnosis image of the diagnosis apparatus pertaining to the first embodiment;

FIG. 14 is the simulation result to indicate the validity of the re-construction algorism of the diagnosis image of the diagnosis apparatus pertaining to the first embodiment;

FIG. 15 is a cross-sectional view illustrating a structure of a probe array in a diagnosis apparatus pertaining to a second embodiment of the present invention;

FIG. 16(a) is a view illustrating real part of a true complex dielectric constant distribution of the target region, in the conventional tomography, for comparison with a hybrid imaging pertaining to a third embodiment of the present invention;

FIG. 16(b) is a view illustrating imaginary part of the true complex dielectric constant distribution of the target region, in the conventional tomography, for comparison with the hybrid imaging pertaining to the third embodiment of the present invention;

FIG. 17(a) is a view illustrating a result when an image is recovered by imaging the complex dielectric constant distribution illustrated in FIG. 16, in the conventional tomography;

FIG. 17(b) is a view illustrating imaginary part of the true complex dielectric constant distribution, as the result when an image is recovered by imaging the complex dielectric constant distribution illustrated in FIG. 16, in the conventional tomography;

FIG. 18 (a) is a view illustrating real part of a true complex dielectric constant distribution of the target region, describing validity of the hybrid imaging pertaining to the third embodiment;

FIG. 18(b) is a view illustrating imaginary part of the true complex dielectric constant distribution of the target region, describing validity of the hybrid imaging pertaining to the third embodiment;

FIG. 19(a) is a view illustrating real part of a true complex dielectric constant distribution, as a result when an image is recovered by imaging the complex dielectric constant distribution illustrated in FIG. 18, in the hybrid imaging pertaining to the third embodiment;

FIG. 19 (b) is a view illustrating imaginary part of the true complex dielectric constant distribution, as the result when the image is recovered by imaging the complex dielectric constant distribution illustrated in FIG. 18, in the hybrid imaging pertaining to the third embodiment;

FIG. 20(a) represents a distribution of dielectric constant, illustrating a distribution of material constant in an original target region, in order to describe a fact that the calculation of the distribution of material constant (tomographic image) is converged, according to the hybrid imaging pertaining to the third embodiment;

FIG. 20(b) represents a distribution of conductivity, illustrating the distribution of material constant in the original target region, in order to describe the fact that the calculation of the distribution of material constant (tomographic image) is converged, according to the hybrid imaging pertaining to the third embodiment;

FIG. 21 is a view describing a state in which a position of an abnormal cell is specified by measuring a reflection from the target region and measuring an energy distribution, in a frequency-space beamforming via a multi-static radar, in the hybrid imaging pertaining to the third embodiment;

FIG. 22(a) represents a distribution of dielectric constant that is obtained by calculation when position information of an abnormal cell is known, illustrating that a tomographic image can be determined, according to the hybrid imaging pertaining to the third embodiment.

FIG. 22 (b) represents a distribution of conductivity that is obtained by the calculation when the position information of the abnormal cell is known, illustrating that the tomographic image can be determined, according to the hybrid imaging pertaining to the third embodiment;

FIG. 23 (a) illustrates that the distribution of dielectric constant diverges, in a calculation when position information of an abnormal cell is not known, for a comparison;

FIG. 23 (b) illustrates that the distribution of conductivity diverges, in the calculation when the position information of the abnormal cell is not known; and

FIG. 24 is a cross-sectional view describing the conventional microwave imaging method.

### DESCRIPTION OF EMBODIMENTS

The first to third embodiments of the present invention will be described below with reference to the drawings. In the descriptions of the following drawings, the same or similar reference numerals are assigned to the same or similar portions. However, the drawings are diagrammatic, and attention should be paid to a fact that the relations between thicknesses and plan view dimensions, the configuration of the apparatus and the like differ from the actual data. Thus, the specific thicknesses and dimensions should be judged by considering the following descriptions. Also, even between the mutual drawings, the portions in which the relations and rates between the mutual dimensions are different are naturally included. Also, the first to third embodiments as described below exemplify the apparatuses and methods for embodying the technical ideas of the present invention, and in the technical ideas of the present invention, the materials, shapes, structures, arrangements and the like of configuration parts are not limited to the followings. Various changes can be added to the technical ideas of the present invention, within the technical range described in claims.

### First Embodiment

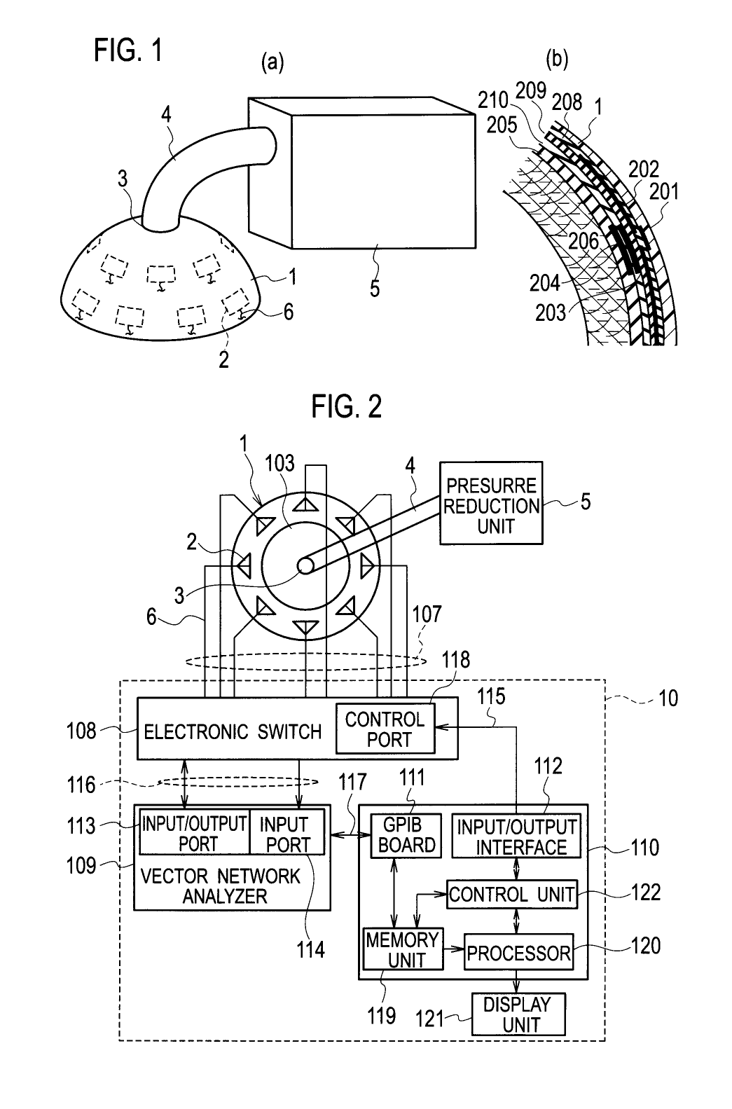

As illustrated in FIGS. 1 (a) and 2, a diagnosis apparatus pertaining to a first embodiment of the present invention encompasses a probe array (1, 2) made of material having electromagnetic characteristic identical to the electromagnetic characteristic of a target object to be measured, the probe array (1, 2) embraces a receptacle 1 having a semispherical inner wall surface, and a plurality of probes 2, arranged along the inner wall surface, the plurality of probes 2 carry out electrical measurement of a target region in the target object, a fixing mechanism(3, 4 and 5) configured to cover whole of the target region with the probe array (1, 2), bringing skin of the target region into close contact with the inner wall surface, so as to fix a relative position between the target region and the probe array (1, 2), and a measuring, controlling and analyzing mechanism 10 configured to control the plurality of probes 2, to execute the electrical measurement, and to analyze a data based on the electrical measurement so as to detect an abnormal cell in the target region.

As illustrated in FIG. 1 (a), in the receptacle 1, the semi-spherical inner wall is made of resin and the like. In the diagnosis apparatus pertaining to the first embodiment, each of the plurality of probes 2 is implemented by an antenna 2 for emitting the electromagnetic wave to the target region to be measured, and the plurality of antennas 2 are arranged on the inner wall surface of the receptacle 1 so that a UWB radar is implemented. The antenna 2 is made of material having an average dielectric constant and dielectric loss of tissues of the target object, and the antenna 2 is the antenna having a flat or conformal multilevel structure. As shown FIG. 1 (a), the fixing mechanism (3, 4 and 5) includes an evacuation unit (4, 5) connected to an evacuation port 3 that is placed near the apex of the probe array (1, 2). As the target region is evacuated by the evacuation unit (4, 5), the skin of the target region is brought into close contact with the inner wall surface of the probe array (1, 2). The evacuation unit (4, 5) encompasses a tubular exhaust tube 4 connected to the evacuation port 3, and a pressure reduction unit 5, such as an aspirator and the like, which is connected to the exhaust tube 4, and the pressure reduction unit 5 decompresses the inner wall side of the probe array (1, 2). The plurality of probes 2 transmit and receive the electromagnetic waves such as the microwaves and the like, and a plurality of input/output cables 6 are extracted from the plurality of probes 2, respectively. A pullout port of the input/output cable 6 is hermetically sealed with resin and the like so that air is not leaked.

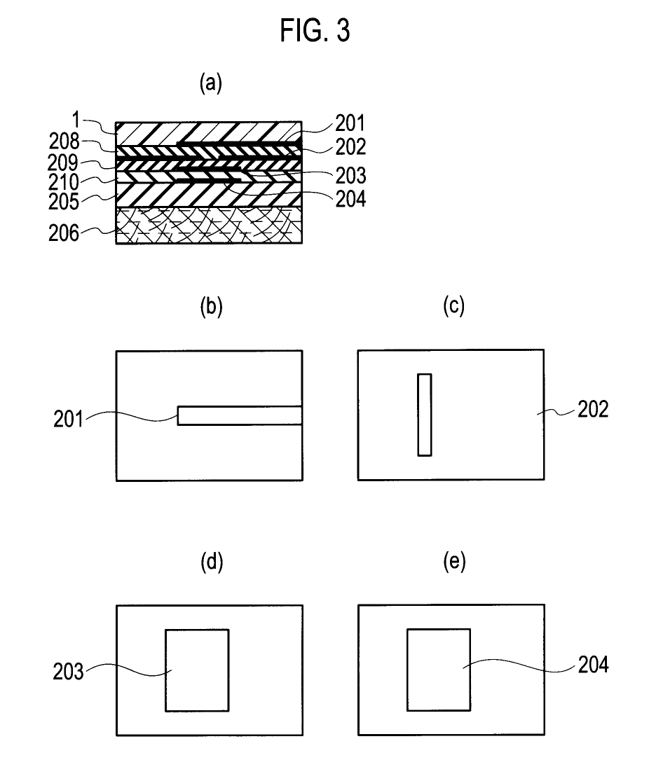

The probe array (1, 2) in the diagnosis apparatus pertaining to the first embodiment can be used to accomodate the whole of the breast as the target region for measurement and screen for the early-stage breast-cancer. For this reason, each of the antennas 2 as the probe of the present invention can employ a stack patch antenna configuration with slot-power-feeding T structure, implemented by four-level configuration, for example, as illustrated in FIGS. 1 (b) and 3. For example, the antenna 2 can be implemented by a dielectric substrate 205 having a thickness of 1.27 millimeters and dielectric constant of 10.2, a dielectric substrate 210 having a thickness of 0.8 millimeter and dielectric constant of 2.2, which is placed on the dielectric substrate 205, a patch layer 203 laminated on a part of an upper surface of the dielectric substrate 210, a patch layer 204 laminated beneath a part of a lower surface of the dielectric substrate 210, a dielectric substrate 209 having a thickness of 1.92 millimeters and dielectric constant of 10.2, being placed on the dielectric substrate 210, a slotted layer 202 laminated on an upper surface of the dielectric substrate 209, a dielectric substrate 208 having a thickness of 0.64 millimeter and dielectric constant 10.2, being placed on the dielectric substrate 209, and a strip line film 201 laminated on an upper layer of the dielectric substrate 208. The value of the dielectric constant of 10.2 is approximately equal to dielectric constant of 9.8 of the fatty tissue of breast.

An output end of the strip line film 201 is connected through a connector or the like to an input/output cable 6, as illustrated in FIG. 1 (a). The strip line film 201 and the patch layers 203, 204 are electromagnetically coupled through the slotted layer 202. Typically, planar sizes of the patch layer 203 and the patch layer 204 are different. The antennas 2 are embedded in the inner wall of the receptacle 1 in a configuration such that a part of the dielectric substrate 205 can be exposed, and the matching is achieved in a state in which the antennas 2 are brought into close contact with the skin of the surface of the target region (breast) 206 for measurement. With such configuration, because the situation of the antenna is electromagnetically equivalent to a state that the antenna is located inside the breast, the electromagnetic wave can be efficiently transmitted into the breast tissue.

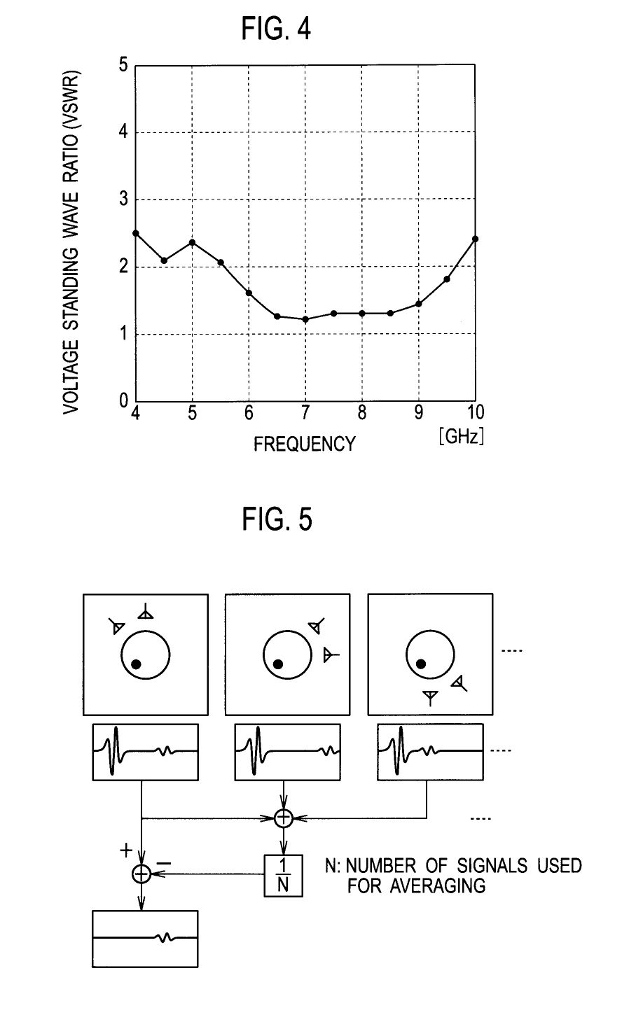

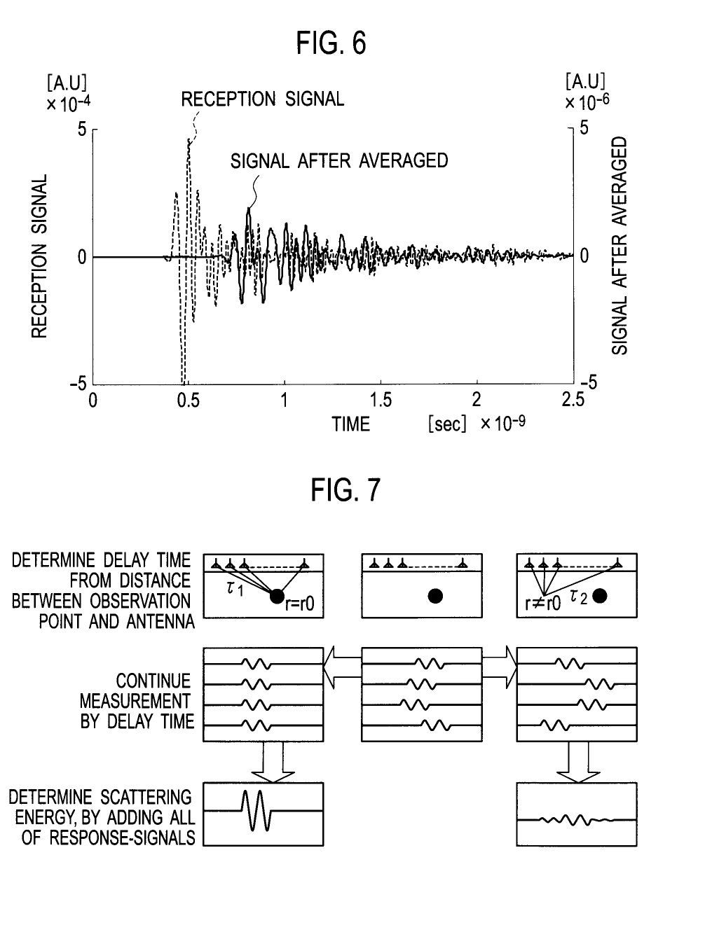

As mentioned above, FIG. 4 represents a voltage standing wave ratio (VSWR) of the antenna 2 that is calculated under the use condition exemplified with reference to FIG. 3. As illustrated in FIG. 4, VSWR<2.5 can be confirmed to be achieved at a frequency between 4 and 10 GHz. With the use of the antenna 2 designed under the assumption of the close contact with the skin, the electromagnetic wave is efficiently transmitted into the target region (breast) 206 for measurement. Thus, the antennas 2 and the target region (breast) 206 for measurement are not required to be immersed into the matching medium.

As illustrated in FIG. 2, the measuring, controlling and analyzing mechanism 10 includes an electronic switch 108 for controlling the driving operations of the plurality of antennas 2, a vector network analyzer 109 for analyzing signals from the plurality of antennas 2, after controlling the driving operations of the plurality of antennas 2 via the electronic switch 108, a controlling operating unit 110 for controlling the switching operation of the electronic switch 108, and a display unit 121 that is connected to the controlling operating unit 110 and displays a measurement condition, a measured-result and the like. As the controlling operating unit 110, it is possible to use a personal computer (PC), various microprocessors and the like.

The electronic switch 108 includes a control port 118. The control port 118 and the controlling operating unit 110 are connected through a cable 115. The electronic switch 108 is further connected through coaxial cables 107 to the probe array (1, 2). The coaxial cables 107 are implemented by the plurality of input/output cables 6 extracted from the plurality of antennas 2. The vector network analyzer 109 includes an input/output port 113 and an input port 114. Each of the input/output port 113 and the input port 114 is connected through a coaxial cable 116 to the electronic switch 108.

The controlling operating unit 110 includes a GPIB board 111 for the mutual connection to the vector network analyzer 109 through a GPIB cable 117, an input/output interface 112 connected to the electronic switch 108 through the cable 115, a memory unit 119 for storing the various parameters with regard to the measurement, the measured-results and the like, a processor 120 for carrying out the various calculations necessary for the measurement and the imaging, and a control unit 122 for controlling the driving operations of the respective units in the controlling operating unit 110, the electronic switch 108 and the vector network analyzer 109.

The diagnosis apparatus pertaining to the first embodiment is used so as to make a position of the evacuation port 3 coincident with a position of the nipple of a person under diagnostic-test, the probe array (1, 2) is put on the target region (breast) 206 for measurement, and evacuation of air is carried out by the pressure reduction unit 5. Since air between the probe array (1, 2) and the gap of the target region (breast) 206 for measurement is evacuated by the pressure reduction unit 5, skin of the target region (breast) 206 for measurement is brought into close contact with the inner wall of the probe array (1, 2) so that the target region (breast) 206 for measurement can be formed into a semi-spherical shape, and thereby, the target region (breast) 206 for measurement is brought into close contact with the plurality of antennas 2. The positional relationship between the plurality of antennas 2 and the surface of the skin of target region (breast) 206 for measurement becomes constant, and the averaging process described with reference to FIG. 5 enables the removal of the large reflection from the skin and enables the response signal from a tumor to be clearly obtained. The shape of the target region (breast) 206 for measurement is mechanically formed. Thus, a person under diagnostic-test is not required to lie in the prone posture (or face-down posture) and make the target region (breast) 206 for measurement droop. Hence, persons under diagnostic-test can receive the inspection while standing. Moreover, in order to correspond to the personal difference in the sizes of the target region (breast) 206 for measurement, a plurality of semispherical probe arrays (1, 2) whose radii are different are prepared in advance, and a specific probe array (1, 2) can be elected so as to mate with each of the sizes of the target region (breast) 206 for measurement.

That is, according to the diagnosis apparatus pertaining to the first embodiment, the received signal is not required to be modified on the basis of the measured distance, and a system for measuring a distance between the target region (breast) and the probe is removed. In the diagnosis apparatus pertaining to the first embodiment, since semispherical receptacles 1 having different radius are prepared so as to facilitate the adoption to various sizes due to personal difference, the breast does not droop, which enables the application to even a person under diagnostic-test whose breast is small. The preparation for the plurality of semispherical receptacles 1 each having the different radius enables the provision of the advantageous effect in which the discomfort feeling caused by the sucking of the target region (breast) 206 for measurement can be reduced. Also, different from the methodologies disclosed in PTLs 2 and 3, since the sensors for the transmission/reception are integrated on the receptacle, it is possible to achieve the advantageous effect in which the movement of a person under diagnostic-test is free.

Here, a configuration of a probe array (1, 2) having N antennas 2-1, 2-2, . . . , 2-N is assumed, and under the assumption, operations of the electronic switch 108, the vector network analyzer 109 and the controlling operating unit 110 will be described.

As described below, the control unit 122 in the controlling operating unit 110 feeds a control signal for connecting the input/output port 113 and the input port 114 of the vector network analyzer 109 and the two probes selected from the plurality of antennas 2, respectively and sequentially, to the electronic switch 108.

The controlling operating unit 110 feeds a command for connecting the input/output port 113 of the vector network analyzer 109 to the first antenna 2-1 in the probe array (1, 2), and a command for connecting the input port 114 to the second antenna 2-2 in the probe array (1, 2), to the electronic switch 108. At the time of the completion of the connection operations in the electronic switch 108, the vector network analyzer 109 feeds a sweep signal in a predetermined frequency range from the input/output port 113 and transmits from the first antenna 2-1. The vector network analyzer 109 receives a reception signal from the antenna 2-2 and measures a propagation loss A₁₂(f) and a propagation phase P₁₂(f) between the first antenna 2-1 and the second antenna 2-2. The propagation loss and the propagation phase indicate the response signal to frequencies, in which frequency is swept. The measured-results are transferred through the GPIB cable 117 to the controlling operating unit 110 and stored in the memory unit 119 in the controlling operating unit 110.

Next, the controlling operating unit 110 feeds a command for connecting the input/output port 113 in the vector network analyzer 109 to the first antenna 2-1, and a command for connecting the input port 114 to the third antenna 2-3, to the electronic switch 108. At the time of the completion of the connection operations in the electronic switch 108, the vector network analyzer 109 measures a propagation loss A₁₃(f) and a propagation phase P₁₃(f) between the first antenna 2-1 and the third antenna 2-3. The measured-results are sent through the GPIB cable 117 to the controlling operating unit 110 and stored in the memory unit 119 in the controlling operating unit 110. The above operations of connecting the input/output port 113 to the first antenna 2-1, and connecting the input port 114 to a still next antenna, for measuring each of propagation losses and propagation phases, are repeatedly and sequentially performed against the transmission side of the first antenna 2-1, until the input port 114 is connected to the antenna 2-N.

Next, the controlling operating unit 110 feeds a command for connecting the input/output port 113 in the vector network analyzer 109 to the second antenna 2-2, and a command for connecting the input port 114 to the third antenna 2-3, to the electronic switch 108. At the time of the completion of the connection operations in the electronic switch 108, the vector network analyzer 109 measures a propagation loss A₂₃(f) and a propagation phase P₂₃(f) between the second antenna 2-2 and the third antenna 2-3. The measured-results are sent through the GPIB cable 117 to the controlling operating unit 110 and stored in the memory unit 119 in the controlling operating unit 110.

Next, the controlling operating unit 110 feeds a command for connecting the input/output port 113 in the vector network analyzer 109 to the second antenna 2-2, and a command for connecting the input port 114 to the fourth antenna 2-4, to the electronic switch 108. At the time of the completion of the connection operations in the electronic switch 108, the vector network analyzer 109 measures a propagation loss A₂₄(f) and a propagation phase P₂₄(f) between the second antenna 2-2 and the fourth antenna 2-4. The measured-results are sent through the GPIB cable 117 to the controlling operating unit 110 and stored in the memory unit 119 in the controlling operating unit 110. The above operations of connecting the input/output port 113 to the second antenna 2-2, and connecting the input port 114 to a still next antenna, for measuring each of propagation losses and propagation phases, are repeatedly and sequentially performed against the transmission side of the second antenna 2-2, until the input port 114 is connected to the antenna 2-N.

The above series of the operations are repeated sequentially until the controlling operating unit 110 feeds a command for connecting the input/output port 113 in the vector network analyzer 109 to the (N−1)-th antenna 2-(N−1) in the probe array (1, 2), and a command for connecting the input port 114 to the N-th antenna 2-N in the probe array (1, 2), to the electronic switch 108, and the vector network analyzer measures the propagation loss 2A_{N-1 N}(f) and the propagation phase P_{N-1 N}(f) between the (N−1)-th antenna 2-(N−1) and the N-th antenna 2-N, and the measured-results are stored in the memory unit 119 in the controlling operating unit 110.

When all of the data obtaining operations have been completed, the measured data are sent to the processor 120, and the backscattered electric field distribution in the imaging range is calculated. The processor 120 displays the calculated result as the diagnosis image on the display unit 121.

The imaging algorism is provided with two processes of a preprocess for removing the artifacts such as the reflection on the skin and the like and a frequency-space beamforming for determining the backscattered power for each pixel in a target region

Data of the propagation loss A_{ab}(f) and the propagation phase P_{ab}(f) between the antenna 2-a and the antenna 2-b (a=1, 2, . . . , N−1, b=1, 2, . . . , N, a<b), which are stored in the memory unit 119, are rewritten to complex signals and then inverse-Fourier-transformed so as to be transformed into time domain signals d_{ab}(t). Since the probes and the skin are in close contact, the response signals from the skin are received in the same way by all of probes. So, the reception signals from two probes having the same positional relationship are averaged, and the averaged signal is assigned as a calibration signal, and the calibration signal is subtracted from the reception signal from each of probes. The subtraction process can remove the signals ascribable to reflections from the skin, which are included in the same way in all of the reception signals from the probes having the same positional relationship. FIG. 5 describes the above process. The above process is performed on all of the combinations of the probes, and the reflection signals from the skin are removed from all of the reception signals.

In the conventional ultrasonic diagnosis apparatus, the imaging is carried out on the basis of “a delay-and-sum (DAS) algorism”. FIG. 7 is an explanatory diagram illustrating the scheme of DAS algorism. In the DAS algorism, the response times received from another plurality of transmission/reception probes located at different positions from the position of a specific transmission/reception probe are calibrated respectively by a propagation delay corresponding to the distance between the specific transmission/reception probe and any pixel located in the target region, and the calibrated response times are added. When an existence of tumor at that point emits large backscattered waves, because the response times of the backscattered response signals from the respective probes are synchronized, when the response signals are added, a large response signal is obtained. When the tumor does not exist at that point, because the response times of the backscattered response signals are not synchronized, even if response signals are added, the large response signal cannot be obtained. The above process is performed on the pixels in the entire target region, and the three-dimensional backscattered-power distribution diagram is prepared. In the DAS, the frequency characteristics of the medium are not considered. A method of considering the frequency characteristics of the medium and using a weight, at which the array gain becomes one at a specified pixel, and then carrying out the directivity synthesis in the frequency-space domain is “the microwave imaging via space-time (MIST) beamforming approach” disclosed in NPL 1.

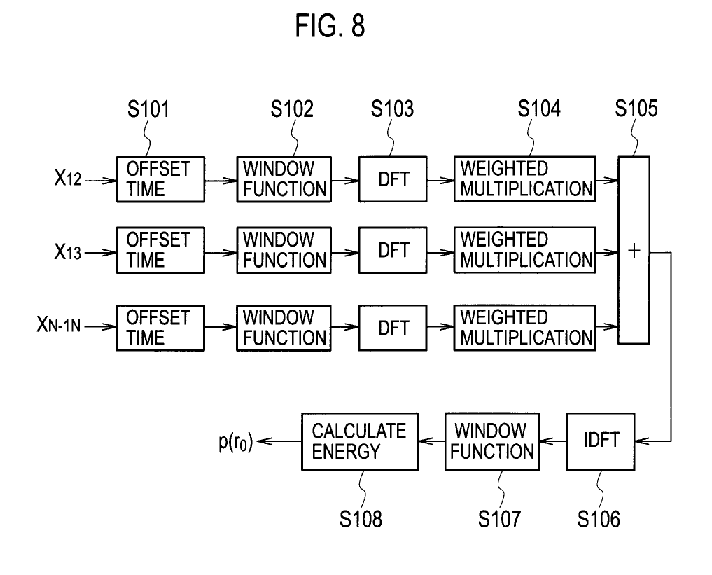

In NPL 1, the use in the monostatic radar in which the transmission/reception are carried out through the probes located at the same position is assumed, and the application in the multi-static radar is not considered. In the present invention, MIST beamforming approach is modified to be able to be applied to the multi-static radar, and the imaging is carried out. The process of the MIST beamforming approach will be described below with reference to FIG. 8 showing the processing procedure at any position rₒ in the target region.

Preliminarily, the control unit 122, after transmitting a measurement start signal to the vector network analyzer 109 and receiving a measurement completion signal of the vector network analyzer 109, reads out the measured-results of the propagation loss and the propagation phase. The read out propagation loss and propagation phase are converted into the complex signals in the processor 120, and then inverse-Fourier-transformed into the time domain signals. Signal transmitted from the i-th probe and received by the j-th probe is defined as xᵢⱼ[n] (n is discrete time).

At first, in Step S101, xᵢⱼ[n] is delayed by a propagation delay time of a sample located at an integer number of sample location (nᵢⱼ[rₒ]=nₐ−τᵢⱼ(rₒ)). Here, τᵢⱼ(rₒ) is propagation delay time in unit of sample interval Tₐ at rₒ. Also,

$$\begin{matrix}[ Eq . 1 ] & \\ {n}_{a} = {max}_{i , {r}_{0}} {τ}_{ij} ( {r}_{0} ) & ( 1 )\end{matrix}$$

nₐ is the maximum propagation delay in the target region.

Next, in Step S102, a next window function is multiplied in order to remove a clutter located prior to nₐ.

$$\begin{matrix}[ Eq . 2 ] & \\ g [ n ] = \{ \begin{matrix}1 : n > {n}_{a} \\ 0 : otherwise\end{matrix} & ( 2 )\end{matrix}$$

In Step S103, the above signal is transformed into the frequency domain, and in Step S104, the beamforming is carried out in the frequency-space domain. A weight Wᵢⱼ[l] of a beamformer, having a linear phase response, with response signal at amplitude of one is represented by the following equation.

$$\begin{matrix}[ Eq . 3 ] & \\ {W}_{ij} [ l ] = \frac{I ( {ω}_{l} ) {\hat{S}}_{ij} ( {r}_{0} , {ω}_{l} ) {e}^{{jω}_{l} {τ}_{o} {T}_{s}}}{\vert I ( {ω}_{l} ) {\hat{S}}_{ij} ( {r}_{0} , {ω}_{l} ) \vert ( 1 + \vert I ( {ω}_{l} ) \vert {\sum }_{i , j = 1}^{N} {\hat{S}}_{ij} ( {r}_{0} , {ω}_{l} ) )} \\ 1 \leq i , j \leq N , i \neq j , 1 \leq l \leq M & ( 3 )\end{matrix}$$

Where, ωₗ is the l-th frequency, I[ωₗ] is the spectrum of transmission signal,

Ŝᵢⱼ(rₒ,ωᵢ)

is the response signal, in which a phase shift associated with the propagation delay is removed from the multi-static radar response at the position rₒ of the l-th frequency, between the i-th probe and the j-th probe, τₒ=(N−1)/2 is the average propagation delay of the beamformer, and M is the value of discrete frequency.

In Step S105, an output in frequency domain of the beamformer is determined. The output in frequency domain is represented by the following equation.

$$\begin{matrix}[ Eq . 4 ] & \\ y ( {ω}_{l} ) = {\sum }_{i , j - 1}^{N} {X}_{ij} ( {r}_{0} , {ω}_{l} ) {W}_{ij}^{*} ( l ) \\ i \neq j , 1 \leq l \leq M & ( 4 )\end{matrix}$$

Where, Xᵢⱼ(rₒ, ωᵢ) is the received signal of frequency domain at position rₒ of the l-th frequency between the i-th probe and the j-th probe. In Step S106, the output in frequency domain is Fourier-transformed and returned to time domain signal z[n], and in Step S107, the following window function is multiplied in order to calculate the backscattered power, and the portion of the main lobe in the time response signal is taken out.

$$\begin{matrix}[ Eq . 5 ] & \\ h [ {r}_{0} , n ] = \{ \begin{matrix}1 : {n}_{h} \leq n \leq {n}_{h} + {l}_{h} \\ 0 : otherwise\end{matrix} & ( 5 )\end{matrix}$$

Here, let us suppose that the main lobe is located at the portion of [nₕ, nₕ+lₕ]. In Step S108, after the window function is multiplied, energy is calculated so as to define the backscattered power p(r₀) at r₀.

$$\begin{matrix}[ Eq . 6 ] & \\ p ( {r}_{0} ) = {\sum }_{n} {\vert z [ n ] h [ {r}_{0} , n ] \vert}^{2} & ( 6 )\end{matrix}$$

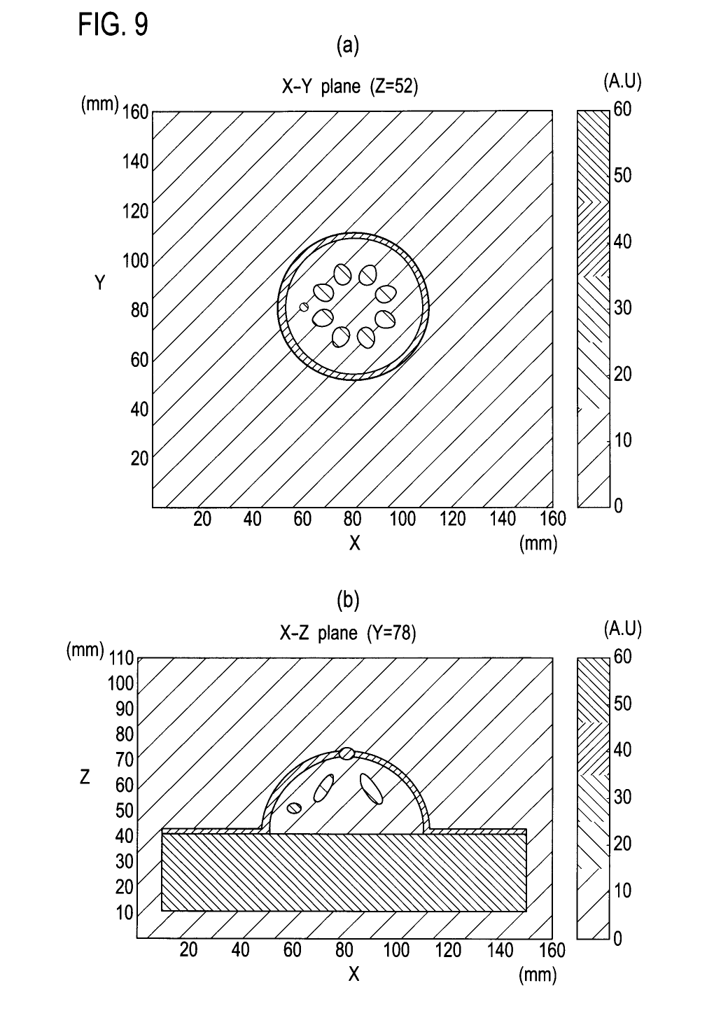

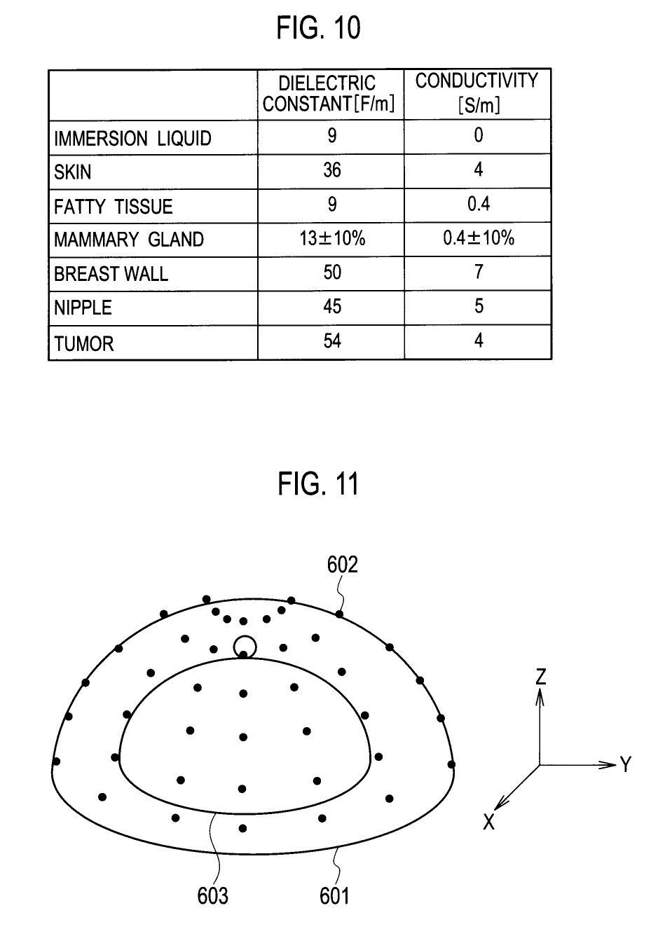

The above calculation is performed on all of the pixels located in the target region. A fact that the algorism of the present invention is superior to the algorism disclosed in NPLs 1 and 2 is indicated by computer simulation. FIGS. 9 to 11 illustrate simulation models. The target region encompasses the skin, fatty tissues, mammary glands, the chest wall, the nipple and a tumor. The dielectric constants and the conductivities of the respective components are collectively illustrated in FIG. 10. As illustrated in FIG. 11, in the probe, 12×6 elements 602 are placed on a hemisphere surface 601. A radius of the tumor is three millimeters, and a thickness of the skin is two millimeters. The pixel is a cube having one side of one millimeter.

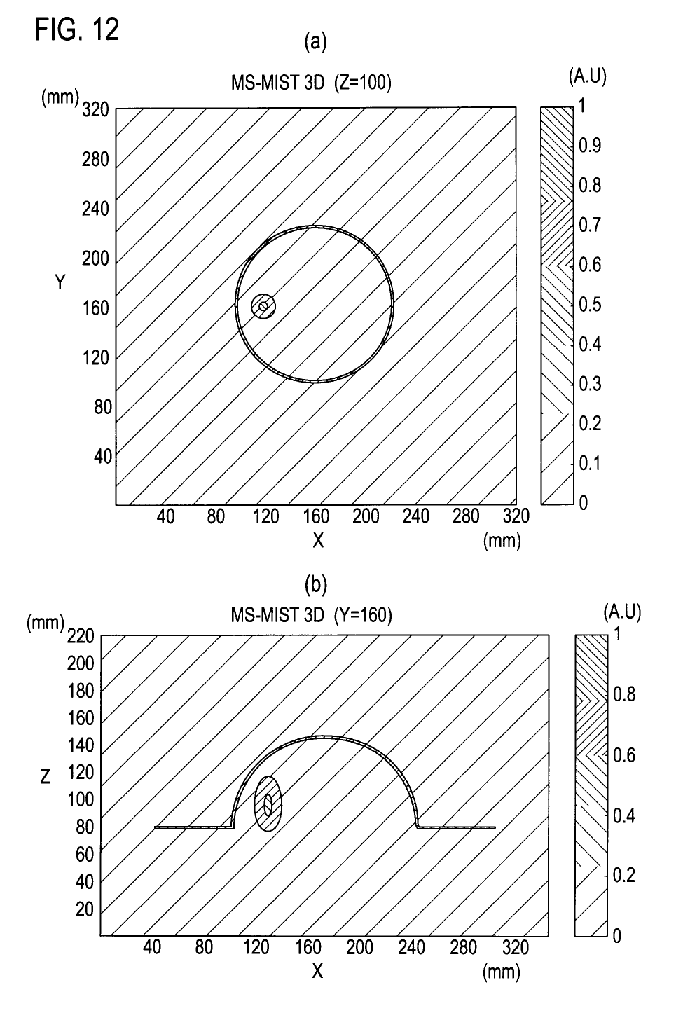

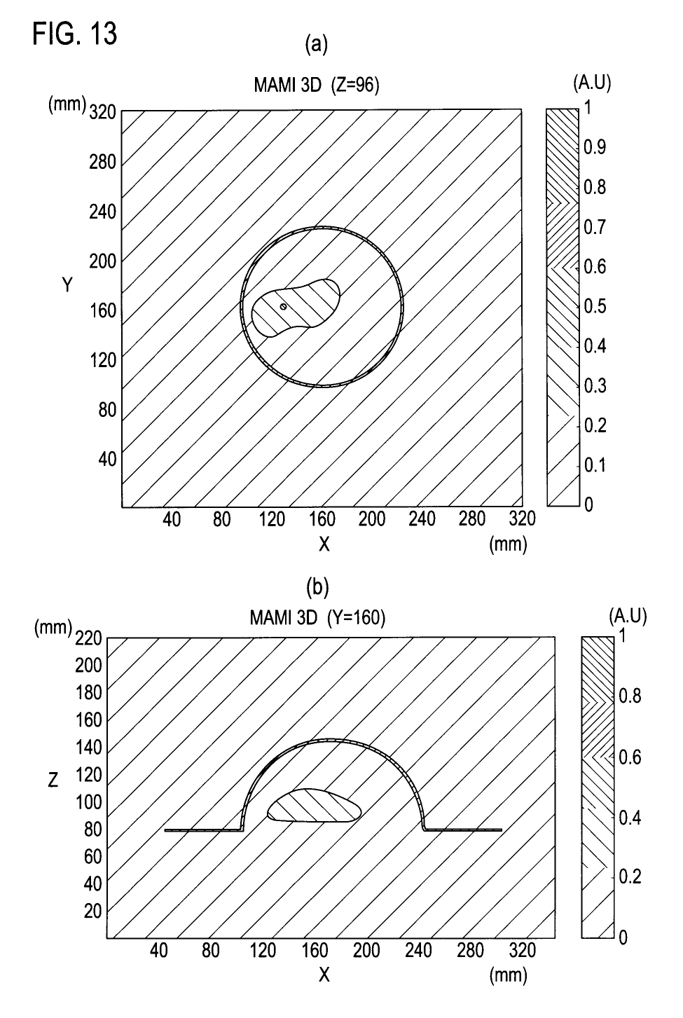

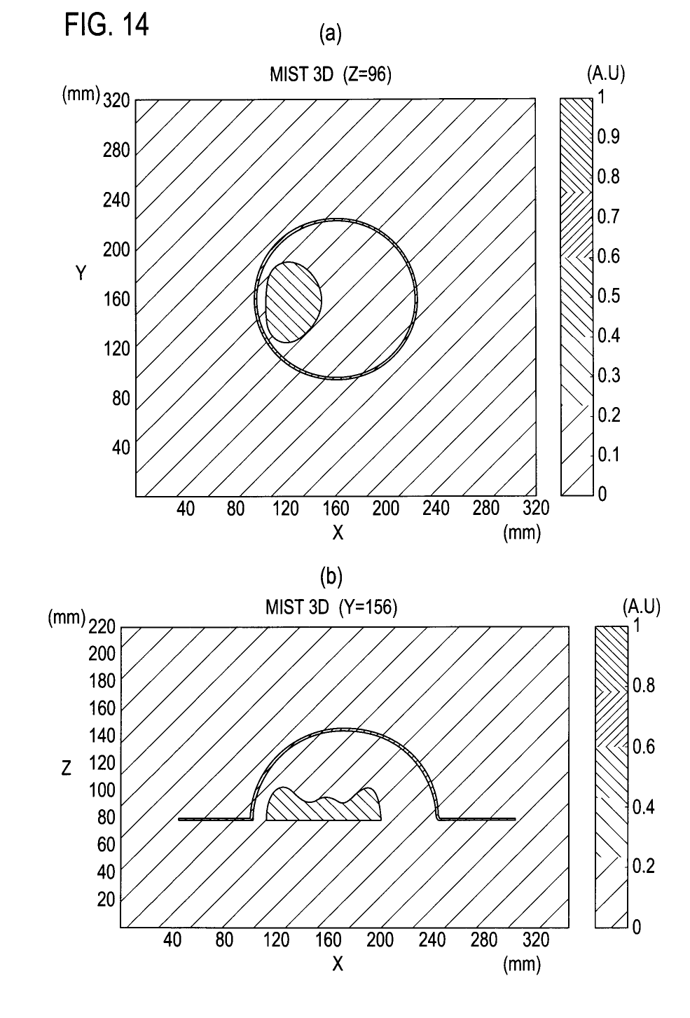

FIG. 12 represents the result of imaging simulation based on the algorism of the present invention, FIG. 13 represents the result of imaging simulation based on the algorism disclosed in NPL 2, and FIG. 14 represents the result of imaging simulation based on the algorism disclosed in NPL 1. In the algorism of the present invention, the tumor is drawn most clearly, and a fake image is minor. With regard to a calculation time per pixel, when a personal computer (PC) operating at 2.2 GHz with Pentium® 4 is used, the calculation time per pixel is 21 seconds by the algorism of the present invention, 186 seconds by the algorism disclosed in NPL 2, and 0.05 seconds by the algorism disclosed in NPL 3. Although the calculation amount is larger than the calculation amount by the algorism disclosed in NPL 1, the calculation speed by the algorism of the present invention lies practically in the allowable range.

As mentioned above, according to the diagnosis apparatus pertaining to the first embodiment, because the matching is achieved in a state in which the antennas 2 as the probe are in close contact with the skin of the object 206 to be measured, and since the electromagnetic waves emitted from the antennas 2 are efficiently transmitted into the target region (breast) 206 for measurement, the antenna 2 and the target region (breast) 206 for measurement are not required to be immersed in matching medium. Also, it is possible to achieve an advantageous effect such that the discomfort feeling is not caused, and that the peripheral contamination ascribable to the droplet of the matching medium is not caused, because the immersion of the target region (breast) 206 for measurement in matching medium is not required.

The UWB radar is one kind of impulse radars and superior in distance resolution. However, because the pulse width of the UWB radar is extremely narrow, it is difficult to carry out the sampling, by directly using an AD converter for the sake of signal process. So, in the diagnosis apparatus pertaining to the first embodiment, the Fourier-transform is used to transform the impulses in the time domain into the frequency domain signals of wideband. The transmission/reception of the impulses is equivalent to the transmission/reception of the frequency sweep signals. The vector network analyzer 109 can measure the propagation and reflection properties while sweeping frequency, the vector network analyzer 109 can be used as a transmitting/receiving unit. Since the vector network analyzer 109 is the general-purpose instrument, the vector network analyzer 109 can achieve the advantageous effect that the vector network analyzer 109 can be easily available and the reliability is high.

In the case of the monostatic radar, the backscattered response signal is determined by the reflection measurement of the vector network analyzer 109. In the case of the multi-static radar, since the transmitting and receiving antennas 2 are separated, the backscattered response signals are determined by the propagation measurement. The multi-static radar having the plurality of reception antennas 2 placed at the different positions can obtain many pieces of backscattered response signal information. Moreover, when the positions of the transmitting antennas 2 are changed and the reception is carried out by changed positions of the transmitting antennas 2, a still larger number of backscattered response signal information can be obtained. In the multi-static radar having the N antennas 2, the D combinations of the transmission/reception can establish at a maximum number of _{N}C₂=N·(N−1)/2. Since the standard vector network analyzer 109 has the input/output of two-port configuration, the simultaneous reception cannot be carried out at a plurality of positions. So, the receiving antennas 2 are sequentially selected, and the reception is carried out by time-sharing system. Similarly, as for the transmission, the transmitting antennas 2 are sequentially selected, and the transmission is carried out by time-sharing system. Therefore, the above electronic switch can be prepared for establishing the system of time-shared transmissions and receptions.

The calculation of the backscattered power distribution in the diagnosis apparatus pertaining to the first embodiment is achieved by applying the beamforming approaches in the frequency-space domain to the multi-static radar response (the propagation loss and the propagation phase) in the frequency domain, a plurality of the multi-static radar responses are obtained by a plurality of transmission/receptions, the number of transmission/receptions is calculated as the combination of n things taken two at a time, with the formula _{N}C₂=N·(N−1)/2. Thus, the parameter that is not uniquely determined is not included in the multi-static radar responses, and the reliability of the imaged result is high. Hence, the diagnosis apparatus pertaining to the first embodiment can achieve the advantageous effect that the calculation amount is minor and the many diagnoses can be endured.

### Second Embodiment

The probe array is not limited to the flat or conformal antenna of the multilevel structure, as described in the first embodiment. A probe array (301, 302) pertaining to a second embodiment of the present invention is molded with semispherical resin layer, similarly to the first embodiment. However, the second embodiment differs from the first embodiment in that a plurality of probes (antennas) 302 are installed in a hollow portion surrounded by inner and outer wall surfaces of a receptacle 301, and matching medium 305, which has the same dielectric constant and conductivity as the fatty layer of the breast, which is assigned as the target region 304 for measurement, is filled.

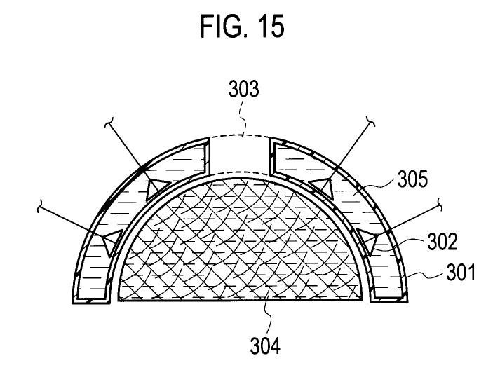

As illustrated in FIG. 15, the probe array (301, 302) includes the receptacle 301 having a semispherical inner wall, a plurality of antennas (probes) 302 arranged in the hollow portion surrounded by the inner and outer wall, and the matching medium 305 filled in the hollow portion.

Although not shown, similarly to the diagnosis apparatus pertaining to the first embodiment, the diagnosis apparatus pertaining to the second embodiment further includes the fixing mechanism (refer to reference numerals 3, 4 and 5 in FIG. 1) for covering the whole of the target region with the probe array (301, 302), bringing the skin of the target region into close contact with the inner wall surface, and fixing the relative position between the target region and the probe array (301, 302), and the measuring, controlling and analyzing mechanism (refer to reference numeral 10 in FIG. 2) for controlling the plurality of probes 302, carrying out the electrical measurement, and analyzing the data based on the electrical measurement, and then detecting the abnormal cell in the target region. In the receptacle 301 pertaining to the second embodiment, as illustrated in FIG. 15, since an evacuation port 303 is placed near an apex, the fixing mechanism pertaining to the second embodiment can evacuate the inner wall side of the receptacle 301 from the evacuation port 303. In such a way that the evacuation port 303 in the receptacle 301 and the position of the nipple of a person under diagnostic-test are coincident, the receptacle 301 is put to cover the whole of the target region (breast) 304, and the evacuation is carried out by the pressure reduction unit (aspirator). With the evacuation process, the skin of the breast 304 is brought into close contact with the inner wall of the receptacle 301, and the breast 304 is semi-spherically formed. Such structure enables the use of the plurality of antennas 302 that do not have the flat or conformal structure. The matching medium 305 having the same dielectric constant and conductivity as the fatty layer of the breast 304 is used, which increases the transmission quantity of the electromagnetic wave into the tissue.

The fixing mechanism is used to evacuate the portion between the probe array (301, 302) and the breast 304, and the inner wall surface of the probe array (301, 302) and the breast 304 are brought into close contact and fixed, which makes the positional relationship between the plurality of antennas 302 and the skin surface of the breast 304 constant. The measuring, controlling and analyzing mechanism is used, and the averaging process described with reference to FIG. 5 is carried out, which can remove the large reflection from the skin. Thus, it is possible to achieve the advantageous effect that the response signal from the tumor can be clearly caught.

Also, the shape of the breast 304 is mechanically formed. Thus, even if the matching medium is used, a person under diagnostic-test is not required to lie in the prone posture (or face-down posture) and make the breast 304 droop. Thus, it is possible to achieve the advantageous effect that a person under diagnostic-test can receive the inspection while standing. The fact that in order to correspond to the personal difference of the size of the breast 304, the plurality of probe arrays (301, 302) whose sizes are different are prepared in advance and the probe array (301, 302) is selectively used on the basis of the size of the breast 304 is similar to the first embodiment.

### Third Embodiment

In the first and second embodiments, the technique that specifies a lesion (or a pathologically changed portion) by using the frequency-space beamforming via the multi-static radar is described. However, a diagnosis apparatus pertaining to a third embodiment of the present invention further includes a tomographic mechanism for performing tomography (or non-invasive imaging) selectively on the periphery of the abnormal cell portion (lesion), after the detection of the abnormal cell (lesion) in the target region, and the hybrid imaging can be carried out in accordance with the hybrid imaging algorism.

According to the hybrid imaging that uses the diagnosis apparatus pertaining to the third embodiment, the complex dielectric constant (the dielectric constant and conductivity) distribution of the target region can be estimated at a higher precision. However, as for the tomographic mechanism in the diagnosis apparatus pertaining to the third embodiment, it is possible to use the known techniques as described in NPL 3 and PTL 1. Or, as the tomographic mechanism pertaining to the third embodiment, it is allowable to configure the means for properly changing the combination of the transmission and reception of the probe 2, inversely calculating the propagation model from the received signal and then estimating the distribution of complex dielectric constant (the dielectric constant and conductivity)—or the tomographic image—of the target region.

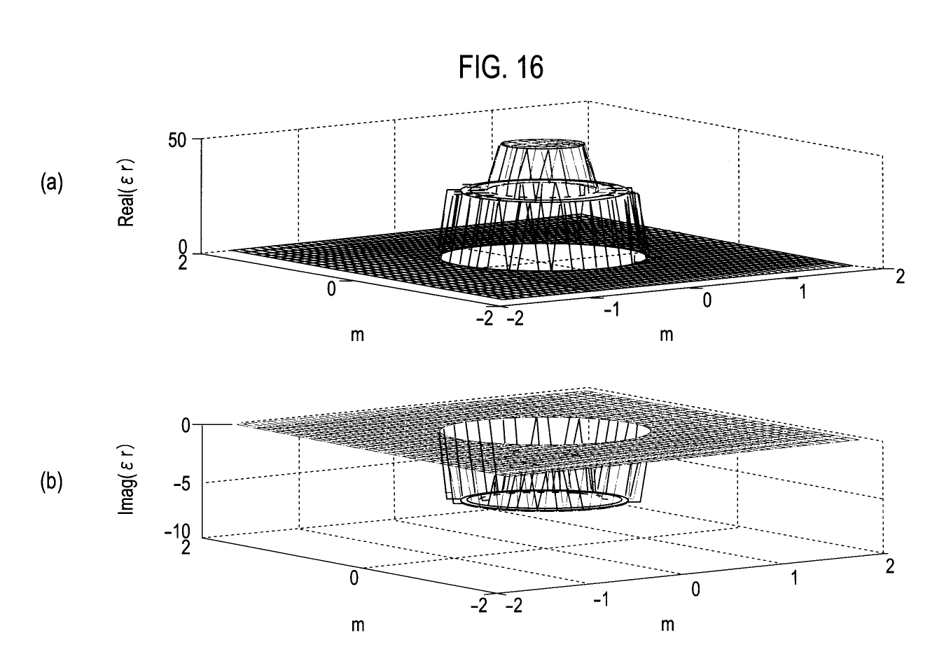

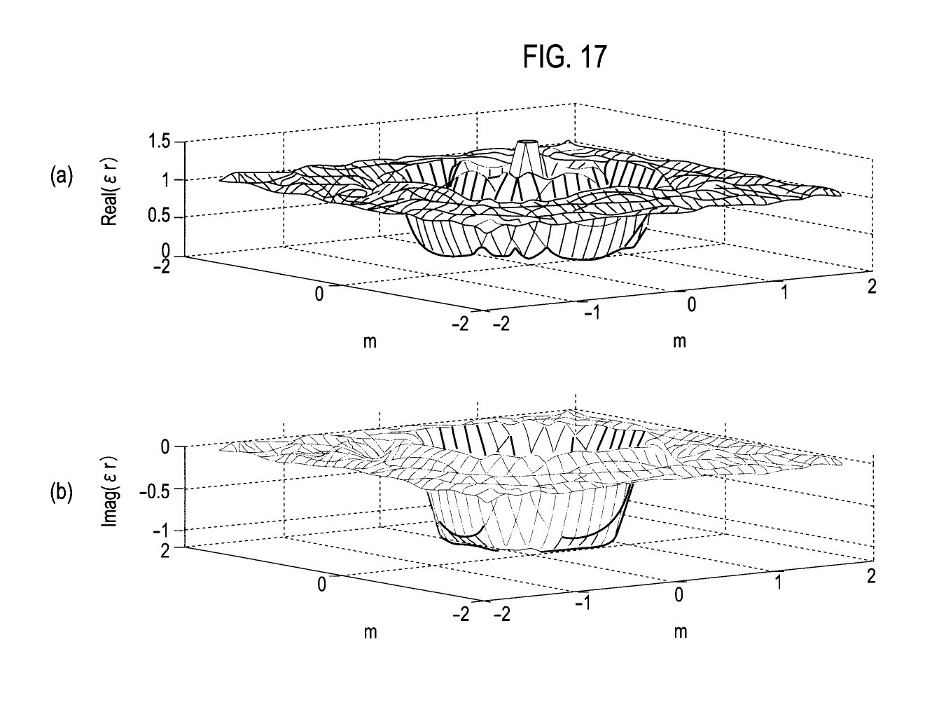

FIGS. 16 to 19 are used to evaluate the validity of the hybrid imaging pertaining to the third embodiment, on the basis of the computer simulation. FIGS. 16 and 17 are the diagrams illustrating the imaged result based on the conventional tomography, for comparison. Then, FIG. 16 represents the true complex dielectric constant distribution inside a certain plane, and FIG. 17 represents the result after the image recovery. In the imaging based on the conventional tomography, the result after the image recovery is known not to exhibit the correct complex dielectric constant.

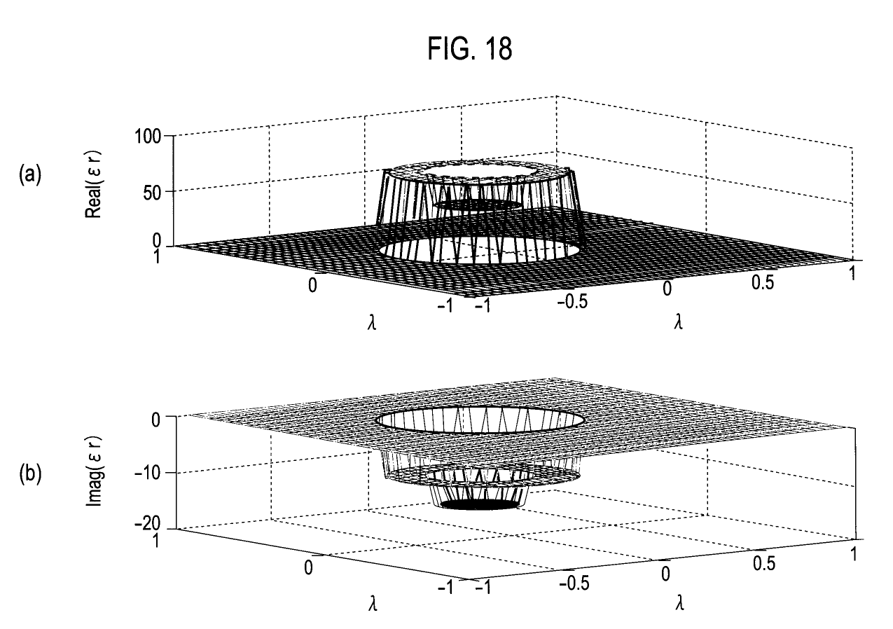

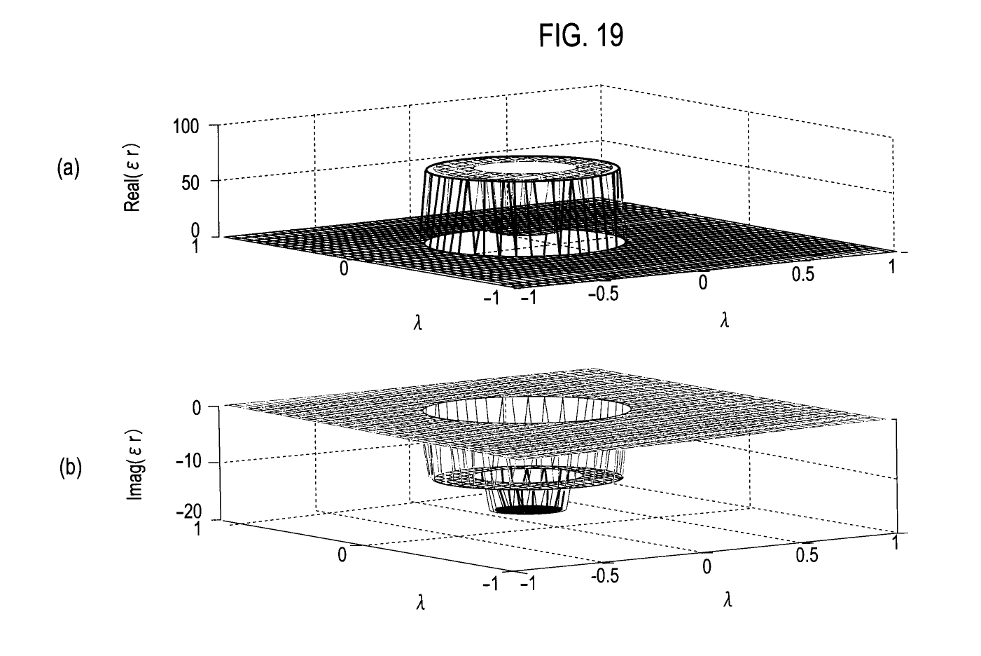

FIGS. 18 and 19 are the diagrams illustrating the result when the hybrid imaging pertaining to the third embodiment, after the position information of the abnormal cell (lesion) is specified and a preliminary knowledge is given, the tomography is carried out. FIG. 18 represents the true complex dielectric constant distribution, and FIG. 19 represents the result after the image recovery. According to the hybrid imaging pertaining to the third embodiment, the result after the image recovery is known to exhibit the correct complex dielectric constant.

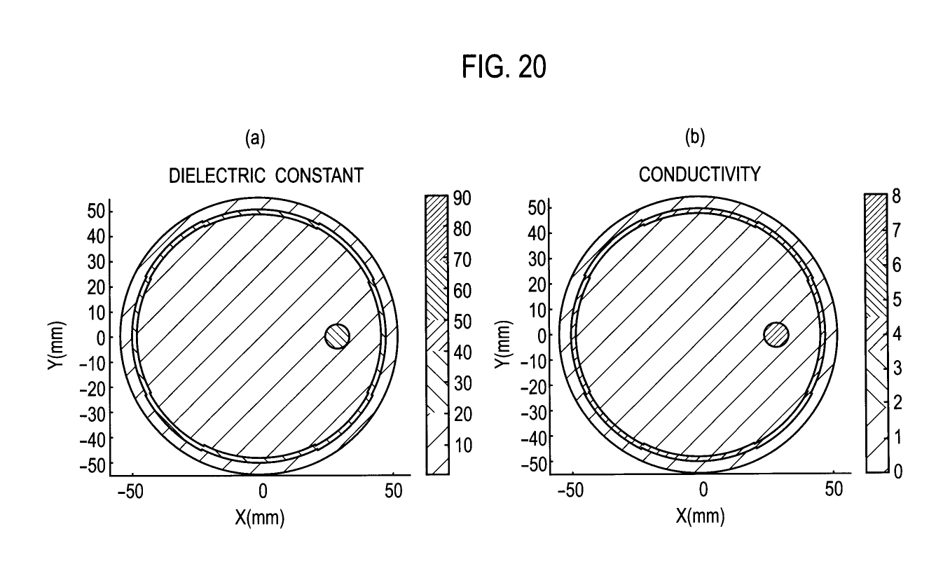

Also, FIGS. 20 to 23 are the diagrams illustrating that, when a distribution of material constant (tomographic image) is determined without any knowledge of position information of the abnormal cell (lesion), the data is diverged, and however, the distribution of material constant (tomographic image) is calculated in a state in which the position information of the abnormal cell (lesion) is known, the data is converged, which enables the determination of the tomographic image.

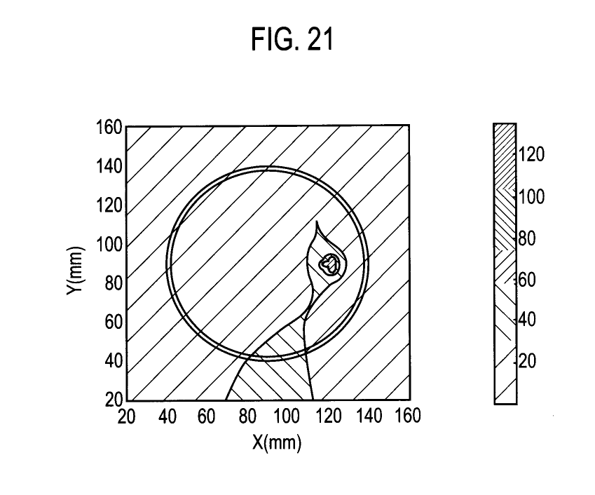

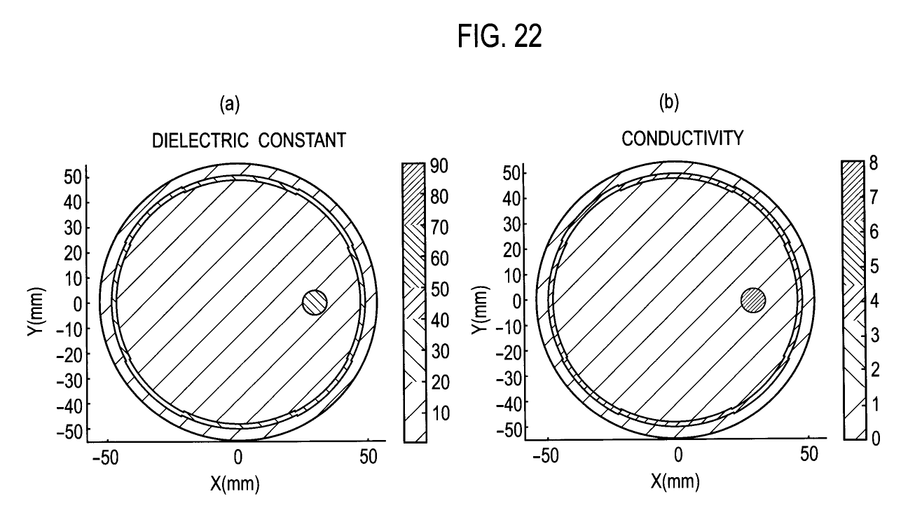

That is, FIG. 20 is the view illustrating the distribution of material constant in the original target region. Then, FIG. 20 (a) represents the distribution of dielectric constant, and FIG. 20 (b) represents the distribution of conductivity. On the other hand, FIG. 21 is the view illustrating that in the frequency-space beamforming via the multi-static radar described in the first or second embodiment, the reflections from the target region are measured to determine the energy distribution and specify the position of the abnormal cell (lesion). As illustrated in FIG. 21, when the position information of the abnormal cell (lesion) is known in the energy distribution, it is known that, when the distribution of material constant (the tomographic image) is calculated, the data is converged, which enables the determination of the tomographic image, as illustrated in FIG. 22. FIG. 22 (a) represents the distribution of dielectric constant obtained from the calculation when the position information of the abnormal cell (lesion) is known, and FIG. 22 (b) represents the distribution of conductivity obtained from the calculation when the position information of the abnormal cell (lesion) is known.

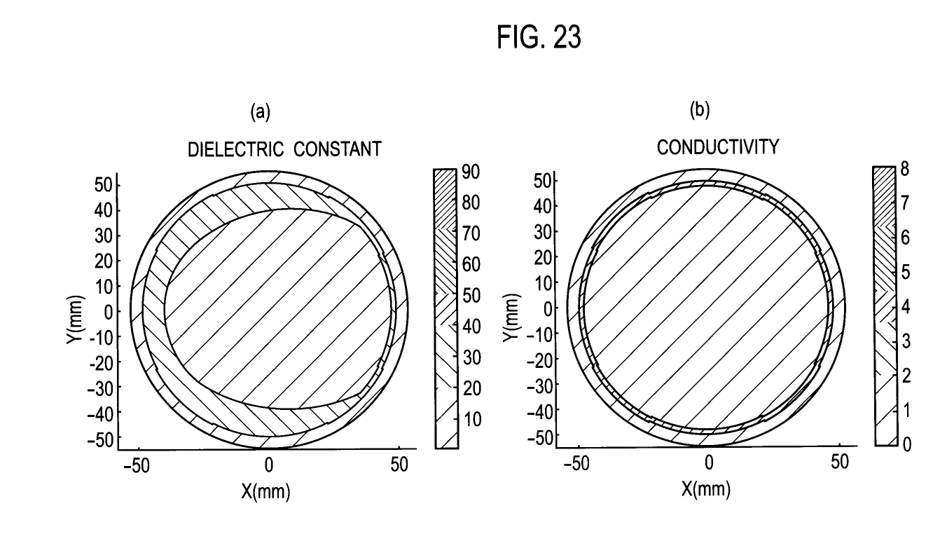

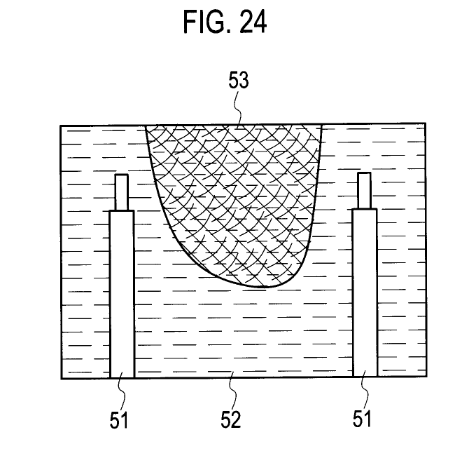

On the contrary, when the distribution of material constant (tomographic image) is determined without any knowledge of position information of the abnormal cell (lesion), as illustrated in FIG. 23, the data is diverged. FIG. 23 (a) represents that the data of the distribution of dielectric constant, which is scheduled to be determined from the calculation, is diverged when the position information of the abnormal cell (lesion) is not known, and FIG. 23 (b) represents that the distribution data of conductivity, which is scheduled to be determined from the calculation, is diverged when the position information of the abnormal cell (lesion) is not known.

In the hybrid imaging pertaining to the third embodiment, since the diagnosis apparatus and the imaging sensor can be commonly shared, the complex dielectric constant distribution of the target region is determined without any reacquisition of data. Also, according to the hybrid imaging pertaining to the third embodiment, there is a merit that the hybrid imaging can be applied to the erasure of the fake image (artifact) generated in the imaging algorism.

### OTHER EMBODIMENT

As mentioned above, the present invention has been described in the light of the first to third embodiments. However, the discussions and drawings that implementing a part of the present disclosure should not be understood to limit the present invention. From the present disclosure, various variations, implementations and operational techniques would be evident for one skilled in the art.

For example, in the first and second embodiments, the algorism based on the measured-results of the propagation loss and the propagation phase between the different probes is described. However, the reflection loss and the reflection phase that are transmitted and received through the same probe may be measured to carry out the directivity synthesis in combination with the measured-results of the propagation loss and the propagation phase. Here, to the above-mentioned processes, next operations are added to the operations of the electronic switch, the vector network analyzer and the controlling operating unit. The controlling operating unit transmits a command for connecting the input/output port of the vector network analyzer to the first probe of the receptacle, to the electronic switch. At the time of the completion of the connection operation in the electronic switch, the vector network analyzer feeds the sweep signal in the predetermined frequency range from the output port, receives the reflection signal from the first probe and measures a reflection loss A₁₁(f) and a reflection phase P₁₁(f) of the first probe.

The measured results are stored in the memory unit in the controlling operating unit. Next, the controlling operating unit transmits a command for connecting the input/output port of the vector network analyzer to the second probe of the receptacle, to the electronic switch. At the time of the completion of the connection operation in the electronic switch, the vector network analyzer measures a reflection loss A₂₂(f) and a reflection phase P₂₂(f) of the second probe. The measured results are stored in the memory unit in the PC. The above operations of connecting the input/output port of the vector network analyzer to the still next probe of the receptacle is repeatedly and sequentially continued until the input/output port of the vector network analyzer is connected to the N-th probe. The above averaging process of is applied to the artifact removal, and the calculation of the backscattered power in the pixel is executed by removing the condition of i≠j of the Eqs. (3) to (5).

Also, in the first and second embodiments, the controlling operating unit 110 may be designed to control the operation of the pressure reduction unit 5 and cooperatively operating the operations of the decompression and the measurement. Therefore, the present invention naturally includes various embodiments that are not disclosed here. Thus, the technical scope of the present invention is determined only by “features to define the invention” recited in claims, which are reasonably supported by the above-mentioned descriptions.

### INDUSTRIAL APPLICABILITY

The diagnosis apparatus and the probe array according to the present invention can be applied to the field of the diagnosis against the abnormal cell, such as the early-stage breast-cancer and the like, which is safe, sure, comfortable and low in cost.

### Reference Signs List

1, 301, 401 . . . Receptacle
2, 302, 402 . . . Probe
3, 303, 403 . . . Evacuation port
4 . . . Exhaust Tube
5 . . . Pressure reduction unit
6 . . . Input/output cable
10 . . . Measuring, controlling and analyzing mechanism
51 . . . Antenna
52, 305 . . . Matching Medium
53, 103, 206, 304, 404, 603 . . . Breast (Target Region for Measurement)
107 . . . Coaxial Cable
108 . . . Electronic Switch
109 . . . Network Analyzer
109 . . . Vector network analyzer
110 . . . Controlling Operating Unit (PC)
110 . . . GPIB Board
112 . . . input/output interface
113 . . . input/output port
114 . . . Input Port
115 . . . Cable
116 . . . Coaxial Cable
117 . . . GPIB Cable
118 . . . Control Port
119 . . . Memory unit
120 . . . Processor
121 . . . Display unit
122 . . . Control unit
201 . . . Strip line film
202 . . . Slotted layer
203, 204 . . . Patch Layer
205, 208, 209, 210 . . . Dielectric Substrate
601 . . . Hemisphere Surface
602 . . . Element

1. A diagnosis apparatus comprising:
a probe array made of material having electromagnetic characteristic identical to the electromagnetic characteristic of a target object, comprising
a receptacle having a semispherical inner wall surface, and
a plurality of probes, arranged along the inner wall surface, configured to carry out electrical measurement of a target region in the target object through signal of frequency-domain;
a fixing mechanism configured to a cover whole of the target region with the probe array, bringing a skin of the target region into close contact with the inner wall surface, so as to fix a relative position between the target region and the probe array; and
a measuring, controlling and analyzing mechanism configured to control the plurality of probes, to apply beamforming in frequency-space domain to multi-static radar response in the frequency-domain, to execute the electrical measurement, and to analyze a data based on the electrical measurement so as to detect an abnormal cell in the target region.

2. The diagnosis apparatus of claim 1, wherein each of the plurality of probes is an antenna configured to emit an electromagnetic wave to the target region.

3. The diagnosis apparatus of claim 2, wherein a plurality of the antennas are arranged on the inner wall surface.

4. The diagnosis apparatus of claim 1, wherein the receptacle is made of material having electromagnetic characteristic identical to the electromagnetic characteristic of the target object.

5. The diagnosis apparatus of claim 2, wherein the receptacle further comprises an outer wall surface opposite to the inner wall surface, and the plurality of the antennas are arranged in a hollow portion surrounded by the inner wall surface and the outer wall surface, and the hollow portion is filled with matching medium.

6. The diagnosis apparatus of claim 1, wherein the fixing mechanism comprises an evacuation unit connected to an evacuation port located near an apex of the probe array,
wherein the target region is evacuated by the evacuation unit so that skin of the target region is brought into close contact with the inner wall surface.

7. The diagnosis apparatus of claim 1, wherein the measuring, controlling and analyzing mechanism comprises:
a vector network analyzer connected to each of the plurality of antennas; and
an electronic switch connected to the plurality of antennas and the vector network analyzer.

8. The diagnosis apparatus of claim 1, wherein the probe array covers a whole of a breast as the target region so as to screen for an early-stage breast-cancer of the breast.

9. The diagnosis apparatus of claim 1, wherein an evacuation hole of the probe array is provided at a location where nipple of the breast as the target region can be inserted.

10. The diagnosis apparatus of claim 7, wherein as a set of the probe arrays, a plurality of semispherical receptacles each has a mutually different radius are prepared, so that one of the receptacle can be elected, the radius of which mates a size of a subject breast,
wherein the elected receptacle accommodate whole of the subject breast.

11. The diagnosis apparatus of claim 2, wherein each of the plurality of antennas is a flat antenna or conformal antenna.

12. The diagnosis apparatus of claim 7, wherein the electronic switch selects one or two of input/output terminals of the plurality of antennas and connects to an input/output port of the vector network analyzer.

13. The diagnosis apparatus of claim 7, wherein the vector network analyzer sweeps frequency and measures a propagation loss and a propagation phase.

14. The diagnosis apparatus of claim 7, wherein the vector network analyzer sweeps frequency and measures a reflection loss, a reflection phase, the propagation loss and the propagation phase.

15. The diagnosis apparatus of claim 7, wherein the measuring, controlling and analyzing mechanism further comprises:
a control unit configured to transmit a control signal, which sequentially connects an input/output port of the vector network analyzer to one of the antenna among the plurality of antennas, and sequentially connects the input/output port of the vector network analyzer to another antenna other than the one of the antenna, to the electronic switch.

16. The diagnosis apparatus of claim 7, wherein the measuring, controlling and analyzing mechanism further comprises:
a control unit which, after transmitting a control signal to the electronic switch, feeds a measurement start signal to the vector network analyzer, and after receiving a measurement completion signal of the vector network analyzer, reads out measured-results of a propagation loss and a propagation phase; and
a memory unit configured to store the measured-results and number of the antennas, which are connected respectively to input/output ports of the vector network analyzer.

17. The diagnosis apparatus of claim 7, wherein the measuring, controlling and analyzing mechanism further comprises:
a control unit which after transmitting the control signal to the electronic switch, feeds the measurement start signal to the vector network analyzer, and after receiving the measurement completion signal of the vector network analyzer, reads out the measured-results of the reflection loss and the reflection phase; and
a memory unit configured to store the measured-results and the number of the antenna connected to input/output ports of the vector network analyzer.

18. The diagnosis apparatus of claim 7, wherein the measuring, controlling and analyzing mechanism further comprises:
a processor configured to synthesize measured-results with a plurality of sets of propagation losses and propagation phases via time-space beamforming, a number of the measured-results, obtained from a plurality of input/output ports associated with one of the input/output port of the vector network analyzer, is equal to the number of the antennas connected to the plurality of input/output ports of the vector network analyzer, so as to determine a backscattered power distribution in the target region; and
a display unit configured to display the backscattered power distribution.

19. The diagnosis apparatus of claim 7, wherein the measuring, controlling and analyzing mechanism further comprises:
a processor configured to synthesize first measured-results with a plurality of sets of propagation losses and propagation phases, a number of the first measured-results, obtained from a plurality of input/output ports associated with one of the input/output port of the vector network analyzer, is equal to the number of the antennas connected to the plurality of input/output ports of the vector network analyzer, and second measured-results with reflection losses and reflection phases, a number of the second measured-results are equal to the number of the antennas connected to the input/output port of the vector network analyzer, via time-space beamforming, so as to determine a backscattered power distribution in the target region; and
a display unit configured to display the backscattered power distribution.

20. The diagnosis apparatus of claim 1, further comprising a tomographic mechanism, after detecting the abnormal cell in the target region, configured to perform tomography selectively on a periphery of the abnormal cell.

21. The diagnosis apparatus of claim 20, wherein the tomographic mechanism, by changing a combination of transmission and reception of the probes, inversely calculates a propagation model from received signals so as to estimate a complex dielectric constant distribution in the target region.
